(19) United States
(12) Patent Application Publication
(10) Pub. No.: US 2009/0137816 A1
(43) Pub. Date: May 28, 2009

(54) Process for the manufacture of diaminofurazan

(76) Inventors: Kurt Baum, Azusa, CA (US); Abdollah Bashir-Hashemi, Mission Viejo, CA (US)

Correspondence Address: JOSEPH E. MUETH, ESQ.;JOSEPH E. MUETH LAW CORPORATION, 100 E. CORSON STREET,, SUITE 300, PASADENA, CA, 91103-3842, US

(21) Appl. No.: 09/738,780

(22) Filed: Dec. 15, 2000

(51) Int. Cl. C07D 271/08

(52) U.S. Cl. 548/125

(57) ABSTRACT

This invention consists of a method for converting diaminoglyoxime to diaminofurazan that can be carried out efficiently at about atmospheric pressure without the need for high pressure containment apparatus.

This invention also consists of a method which comprises converting diaminoglyoxime to diaminofurazan at about atmospheric pressure in the presence of a strong base and/or an organic polar solvent.

### BACKGROUND OF THE INVENTION

Furazan derivatives have been of interest for the construction of rocket propellants and explosives ingredients because the compounds are relatively insensitive and yet provide favorable oxygen balance. The starting point is usually diaminofurazan, and oxidation converts amino groups to nitro groups, and also to azo and azoxy groups. The azo and azoxy groups serve as energetic linking groups that link two or more W furazan rings. Solidyuk, G. D.; Boldyrev, M. D.; Gidaspov, B. V.; Nikolaev. V. D. Zhur. Org. Khim. 1981 17 (4), 861-5. Kulagina, V. O.; Novikova, T. S.; Mel'nikova, T. M.; KhmeInitskii, L. I. Chem. Heterocylic Comp. 1994, 30 (5), 631-5. Kulagina, V. O.; Novikova, T. M.; KhmeInitskii, L. I. Chem. Heterocyclic Comp. 1994, 30(5), 629-30. Sheremetev, A. B.; Kulgina, V. O.; Aleksandrova, N. S.; Dmitriev, D. E.; Strelenko, Y. A.; Lebedev, V. P.; Matyushin, Y. N. Propellants, Explosives, Pyrotechnics 1998, 23, 142-9. Gunasekaran, A.; Trudell, M. L.; and Boyer, J. H. Heteroatom Chem., 1994, 516, 441. Diaminofurazan has also been used as a ballistic modifier to suppress the burn rate and pressure exponent of ammonium perchlorate composite propellants. Stoner, C. E., Jr.; Brill, T. B. Combustion Flame 1991, 83, 302.

The large scale use of furazan-based energetic materials, however, has been restricted because of the difficulty of preparing diaminofurazan. The generation of the furazan ring by the dehydration of diaminoglyoxime at elevated temperatures in aqueous sodium or potassium hydroxide has been known for over a century. Wolff, L. Chem. Ber., 1895, 28, 69. However, the efficient dehydration of diaminoglyoxime required a high temperature sealed tube reaction process. Coburn, M. D. J. Heterocyclic Chem., 1968, 5, 83. Improvements in the synthesis of diaminofurazan have recently been reported using a “simple stainless steel reactor”, but costly equipment would still be needed for large-scale operation. Gunasekaran, A.; Jayachandran, T.; Boyer, J. H.; and Trudell, M. L. J. Heterocyclic Chem., 1995, 32, 1405.

### SUMMARY OF INVENTION

Briefly, this invention comprises of a method for efficiently converting diaminoglyoxime to diaminofurazan which comprises heating diaminoglyoxime at about atmospheric pressure.

More particularly, this invention comprises converting diaminoglyoxime to diaminofurazan at about atmospheric pressure with or without the presence of a strong base.

Further, this invention comprises converting diaminoglyoxime to diaminofurazan at about atmospheric pressure in the presence of a solvent which does not boil at the reaction temperature or without the presence of a solvent.

The present invention eliminates the need for pressure containment equipment and the risks associated therewith.

### DESCRIPTION OF PREFERRED EMBODIMENTS

In the procedure of this invention, a hot solution of diaminoglyoxime and potassium hydroxide in ethylene glycol gave diaminofurazan in 52% yield. The yields have not been optimized, but they are higher than yields that have been obtained in pressure reactions using water as the solvent. A reported yield of 70% (Gunasekaran, A. et al, supra) could not be reproduced by other workers, and the same group later reported 30%: Zelenin, A. K.; Stevens, E. D.; Trudell, M. L. Structural Chem., 1997, 8 (No. 5), 373-377.

Potassium hydroxide was used as the base, but other strong alkali and alkaline earth metal bases, such as sodium hydroxide or calcium hydroxide are also suitable. Ethylene glycol was used as the solvent for the diaminoglyoxime, but other similar solvents or mixtures of solvents with boiling points higher than about 150° C. can be used. The solvent is selected so as to have a boiling point higher than the reaction temperature. Examples are diethylene glycol, polyethylene glycols, glycerol, propanediols, and butanediols. Other polar solvents, such as amides and esters can be used, but care would be needed to determine that hydrolysis of the solvent is significant at the reaction temperature. Work-up consists of diluting the cooled reaction mixture with cold water, and collecting the solid product. The product crystallizes readily from ethylene glycol—water. Other solvents may require more complex work-up because of tendency for the product to separate as an oil. The reaction was carried out by preheating ethylene glycol to 120° C., adding diaminoglyoxime and potassium hydroxide, and then heating the mixture at 170° C. for one hour. The conversion reaction can be carried out at temperatures on the order of from about 100° C. to about 250° C.

The reaction is preferably carried out at atmospheric pressure in an open reactor system. However, it is to be understood that variations from atmospheric up to several atmospheres (about 3-5) or at less than atmospheric by the use of a vacuum are within the scope of the term “about atmospheric pressure” in the practice of this invention which has as its major achievement the elimination of the need for high pressure containment apparatus.

If the ethylene glycol is not preheated before adding the solid reagents, a thick slurry is obtained that is difficult to stir.

In the preferred embodiment, the reaction is depicted as follows:

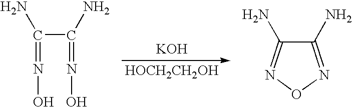

The following Examples are illustrative.

### EXAMPLE 1

Diaminofurazan. In a 500-ml round bottom flask equipped with a mechanical stirrer and a thermometer, ethylene glycol (150 ml) was heated to 120° C., and to this solution were added diaminoglyoxime (50 g, 0.42 mol) and then potassium hydroxide (24 g, 0.42 mol). The reaction mixture was heated at 170° C. for one hour. The clear solution was cooled to room temperature and poured into a mixture of ice (500 g) and water (100 ml). The mixture was shaken for five minutes until solid crystals of diaminofurazan were formed. The precipitate was filtered and washed with 20 ml of cold water and air-dried overnight to give 22 g (52%) of off-white solid: mp 179-181° C. lit Gunasekaran, A. et al, supra, 179-180° C.; ¹H NMR (DMSO-d₆), 5.81 (s) ppm.

### Modified Synthesis of Diaminofurazan

Additional experiments revealed that the use of a base is not required. Similar yields can be obtained simply by heating diaminoglyoxime in a polar solvent such as ethylene glycol. The reaction even takes place by heating diaminoglyoxime neat. The neat reaction appears to yield diaminofurazan in combination with impurities which complicate product purification and requires a subsequent purification step such as dissolving the reaction products in a polar solvent and recovering the purified diaminofurazan by crystallization.

### EXAMPLE 2

A solution of diaminoglyoxime (1.80 g, 15 mmol) in 5.0 ml of ethylene glycol was heated at 165° C. for 30 minutes. The clear solution was then cooled to 25° C. and diluted with 50 ml of cold water. The resulting solution was saturated with sodium chloride and subsequently extracted with ethyl acetate (3×50 ml). The combined organic layer was washed with brine and dried over MgSO₄. Concentration by rotary evaporation gave pure diaminofurazan (780 mg, 52%). ¹H NMR (DMSO-d₆) δ 5.81 ppm. mp. 178-180 C.°

### EXAMPLE 3

In another experiment a neat sample of diaminoglyoxime (50 mg) was heated at 165° C. for 30 minutes; proton nmr of the reaction mixture in DMSO-d₆ showed 70% conversion to diaminofurazan.

1. A method for efficiently converting diaminoglyoxime to diaminofurazan which comprises heating diaminoglyoxime at about atmospheric pressure.

2. A method of claim 1 where the converting of diaminoglyoxime to diaminofurazan is carried out in the presence of a strong base.

3. The method of claim 2 wherein the strong base is an alkali or alkaline earth metal hydroxide.

4. The method of claim 2 wherein the strong base is potassium hydroxide.

5. The method of claim 1 wherein the converting is carried out in the presence of an organic polar solvent.

6. The method of claim 5 wherein the organic polar solvent has a boiling point above about 150° C.

7. The method of claim 6 wherein the organic polar solvent is ethylene glycol.

8. The method of claim 1 wherein the converting is carried out at a temperature of about 100° C. to about 2500° C.
